(19) United States
(12) Patent Application Publication
(10) Pub. No.: US 2005/0277169 A1
(43) Pub. Date: Dec. 15, 2005

(54) Active microcosm: screening antimicrobial producing microorganisms

(76) Inventor: Janet M. Sigmund, Cranford, NJ (US)

Correspondence Address: MERCK AND CO., INC, P O BOX 2000, RAHWAY, NJ, 07065-0907, US

(21) Appl. No.: 11/129,719

(22) Filed: May 13, 2005

Related U.S. Application Data
(60) Provisional application No. 60/571,269, filed on May 14, 2004.

(51) Int. Cl. C12Q 1/18; C12Q 1/04

(52) U.S. Cl. 435/32

(57) ABSTRACT

The Active Microcosm approach offers a new method for detecting antimicrobial producing microorganisms in natural samples prior to plating and culture isolation. The method involves inoculating portions of natural substrates; soil, litter, water etc., onto different agar nutrient media contained in the wells of a standard 96-well membrane filter plate.

### CROSS-REFERENCE TO RELATED APPLICATIONS

This application claims benefit of U.S. provisional application 60/571,269 filed on May 14, 2004.

### BACKGROUND OF THE INVENTION

The traditional approach to find antimicrobial producing microorganisms in screening programs relies on the random selection of populations of microorganisms associated with diverse natural samples. From these populations, cultures are selected, usually on the basis of different morphological or other taxonomic criteria. The selected cultures then are fermented and the fermentation broths assayed for the presence of antimicrobial activity.

The primary disadvantages of this approach are that it is highly labor intensive and inherently inefficient since most of the microorganisms that are examined, routinely do not produce any antimicrobial activity. Consequently, many different natural substrates must be collected, returned to the laboratory, and plated onto different agar media. Very large numbers of the associated microorganisms must then be isolated, fermented and examined to find the very small percentage that are antimicrobial producers.

The agar plug method can be used to screen cultures for antimicrobial activity (Bragulat, et al, Int J Food Microbiol 71(2-3): 139-44 (2001); Smedsgaard, J Chromatogr A. 760(2):264-70 (1997); Mazza, Appl Environ Microbio. 45(6):1949-52 (1983)). In this method, agar plugs are cut manually from Petri dishes on which a culture is grown. First, a natural sample is streaked or diluted onto an agar plate. Typically, one to several isolation media are used for this purpose. Subsequently, single colonies of cultures are randomly selected from this original plate and transferred to another agar plate for purification and growth. An agar plug can be excised from the plate at this point to detect the production of any antimicrobial compound in agar by the culture. The agar plug can be extracted and assayed in a liquid format or the agar plug may simply be placed on the surface of an agar assay plate. This procedure circumvents the scale-up of growth of the culture in liquid prior to assay.

Nishikawa & Ogawa describes screening forest soil isolates directly on an agar plate containing dyes (Appl Environ Microbiol 68(7):3575-81 (2002)). Following incubation of plates, colonies interacting with dyes were picked and purified for further testing. In this manner, cultures producing employs the traditional culture isolation technique of streaking or diluting the natural sample on agar Petri dishes in order to obtain isolates using a limited number of media. Streaking and diluting the soil sample on agar plates is time consuming and thereby limits the number of soil samples that can be screened.

Thus, there is a need to develop an efficient method to screen antimicrobial producing microorganisms. The references cited herein are not admitted to be prior art to the claimed invention.

### SUMMARY OF THE INVENTION

The present invention relates to a method of screening microorganisms. The method comprises inoculating to media a sample containing a plurality of different microorganisms, incubating the inoculated media, and detecting the presence of microorganisms having desired characteristics on the incubated medium with a reporter system. The method can further comprise isolating the detected microorganisms. According to an embodiment of the present invention, the desired characteristics include producing antimicrobials.

In an embodiment, the sample used in the method is selected from the group consisting of soil, litter, and water.

According to an embodiment of the present invention, the media are agar nutrient media. According to a preferred embodiment of the present invention, the media comprise a basal salts mineral medium supplemented with a carbon and/or nitrogen source. According to a further preferred embodiment of the present invention, the agar nutrient media are different agar nutrient media.

According to an embodiment of the present invention, the media are contained in the wells of a 96-well membrane filter plate. According to a preferred embodiment, the media are contained in a plate with a membrane filter side.

According to an embodiment of the present invention, the presence of microorganisms producing antimicrobial is detected by placing the membrane filter side with a reporter system. According to a preferred embodiment of the present invention, the reporter system is agar seeded with a microorganism targeted by the antimicrobial, such as Staphylococcus aureus.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1 Schematic representation of a 24 media array in a microcosm plate

FIG. 2 Microcosm plate inoculated by sprinkling a sample of dry sand

FIG. 3 Close-up of wells of a microcosm plate inoculated by brushing a sample of moist sediment

FIG. 4 Detection of active microcosms; antibiotic containing microcosm wells produce a zone of inhibition vs S. aureus

FIG. 5 Detection of antibiotic producing colonies; (A) colonies on isolation plate (B) overlay with S. aureus—antibiotic producing colonies generate a zone of inhibition.

### DETAILED DESCRIPTION OF THE INVENTION

1. Microcosm

The term ‘microcosm’ refers to a commonly employed technique in microbial ecology where a sample from the environment, usually a soil sample, is studied in the laboratory under conditions closely approximating the environment from which it was removed. Typically, a microcosm consists of a soil sample that is incubated and maintained in a closed container at a specific temperature, atmosphere, and condition (e.g., light, dark, pH). The microcosm is often sampled with time to examine differential effects. The microcosm has proven to be a very useful tool in studies in various fields, including:

a) the transformation of xenobiotics (Deni, & Penninckx, Appl. Environ. Microbiol. 65:4008-13 (1999); Hopkins, et al, Appl. Environ. Microbiol. 59:2277-85 (1993); Jitnuyanont, et al, Biodegradation 12:11-22 (2001); Shi, et al, Appl. Environ. Microbiol. 65:2143-50 (1999));

b) the gene transfer among bacteria (Orus & Vinas, Microb. Drug Resist. 6:99-104 (2000); Sengelov & Sorensen, Curr. Microbiol. 37:274-80 (1998); Vionis, et al, Antonie Van Leeuwenhoek 73:103-115 (1998); Weekers, et al, Appl. Biochem. Biotechnol. 91-93: 219-32 (2001));

c) the fate of genetically engineered bacteria (Awong, et al, Appl. Environ. Microbiol. 56:977-983 (1990); Miyagi, et al, Appl. Environ. Microbiol. 61:2030-32 (1995); Pipke, et al, Appl. Environ. Microbiol. 58: 1259-1265 (1992));

d) the effect of antibiotic producing bacteria on plant pathogens (Ellis, et al, J Appl. Microbiol. 87:454-463 (1999); Rodriguez Ortega, et al, Microbios 106:39-47 (2001); Russo, et al, J Ind. Microbiol. Biotechnol. 27:337-342 (2001); Thrane, et al, FEMS Microbiol. Ecol. 33:139-146 (2000)), and

e) microbial ecology (Almeida, et al, Microb. Ecol. 42:562-571 (2001); Anderson, et al, Microb. Ecol. 42:474-481 (2001); Morris, et al, Appl. Environ. Microbiol. 68:1446-1453 (2002); Wohl & McArthur, Microb. Ecol. 42:446-457 (2001)).

2. Screening Antimicrobial Producing Organisms with Active Microcosm

The present invention provides a method of screening antimicrobial producing microorganisms. The method comprises inoculating to media a sample containing a plurality of different microorganisms, incubating the inoculated media, and detecting the presence of microorganisms having desired characteristics on the incubated medium with a reporter system. As used herein, the method is called “active microcosm”. According to an embodiment of the present invention, the method further comprises isolating the detected microorganisms.

According to a preferred embodiment of the present invention, the desired characteristics include producing antimicrobials.

According to an embodiment of the present invention, the sample is selected from the group consisting of soil, litter, and water.

According to an embodiment of the present invention, the media are agar nutrient media. According to an embodiment of the present invention, the agar nutrient media are different agar nutrient media. According to a preferred embodiment of the present invention, the media comprises a basal salts mineral medium supplemented with a carbon and/or nitrogen source. In general, the final concentration of organic carbon and/or nitrogen source in the medium can be varied between 1 g/L to 4 g/L. Examples of commonly used carbon sources are glycerol, dextrin and soluble starch as well as organic acids. Examples of commonly used nitrogen sources are inorganic, such as NH₄NO₃ or NaNO₃, or organic such as peptone or yeast extract. Antibiotic solutions may also be incorporated into the medium if desired. The choice of media composition can be dependent on the growth of the type(s) of microorganisms of interest to the investigator.

2.1. High Volume Screening

The present invention can be utilized in high volume screening. According to an embodiment of the present invention, the media are contained in the wells of a 96-well membrane filter plate, such as Millipore MultiScreen plate (http://www.millipore.com).

Microplate technology allows miniaturization of the process, making it more efficient in efforts to streamline the drug discovery process. The Millipore MultiScreen plate or a similar 96 well plate is used for rapid and efficient HTS screening, either for antimicrobial detection (Morgan et al, J Appl Microbiol 89(1):56-62 (2000); Dufou, et al, Int J Food Microbiol 85(3): 249-58 (2003); Jewel, et al, J Microbiol Methods 49(1): 1-9 (2002)) or other screening platforms (Tiberghien, et al, J Immunol Methods 223(1):63-75 (1999); Blevit, et al, J Biomol Screen 4(2):87-91 (1999)). Recovery of proteins from solid gel pieces using the Multiscreen plate is also described (Pluska, et al, Proteomics 2(2): 145-50 (2002)).

According to an embodiment of the present invention, one soil sample can be directly placed on the surface of agar or agarose based media contained in the 96 wells of the MultiScreen plate. Each well of the MultiScreen plate can contain a different or same agar or agarose based medium. The soil sample does not need to be streaked across an agar plate, nor does it have to be diluted at this stage. The Multiscreen plate can be filled with agar (or agarose in various amounts up to 275 microliters) using automated equipment such as the Multidrop 384, Quadra or multi-channel automated pipeting systems, thereby eliminating the manual preparation of many agar Petri dishes.

According to a further embodiment of the present invention, all 96 agar or agarose-filled wells in the MultiScreen plate can rapidly and simultaneously be extracted by filtration or centrifugation in order to obtain a liquid sample for further evaluation. Lastly, the standard size of the 96 well plate means that the plate can be moved or manipulated with commercially available or customized robotic systems for automation purposes.

2.2. The Reporter System

The reporter system should be capable of distinguishing the desired organisms and other organisms. When detecting antimicrobial producing microorganisms, for instance, the reporter system can be agar seeded with a microorganism targeted by the antimicrobial. The presence of microorganisms that produce antimicrobial activity is detected by placing the membrane filter side of the wells of the plate in contact with an agar seeded with the targeted microorganism, such as Staphylococcus aureus. Any antimicrobial compounds associated with a well are transported into the agar by diffusion through the membrane filter. After incubation, antimicrobial activity is detected as a clear zone where the growth of the seeded microorganism has been inhibited. The detection of antimicrobial activity from a well suggests the presence of antibiotic producing microorganisms associated with the natural sample present in the well. Conversely, the absence of antimicrobial activity infers the absence of any microorganisms producing activity against the selected reporter system. A microorganism capable of producing the desired activity may be present, but did not grow or produce activity in the well in that particular medium or environment. Thus, by evaluating the presence or absence of antimicrobial activity that is associated with each well when tested against a particular reporter system, one can easily target for further study only those microcosms with the potential to yield antimicrobial producing microorganisms.

2.3. The Utilities of the Present Invention

The present invention allows the recognition and selection of natural samples that in situ appear to harbor microorganisms with the potential to produce antimicrobial activity prior to beginning culture isolation work. Thus, only those samples exhibiting intrinsic antimicrobial production can be selected for subsequent isolation of the producing microorganism(s) while the others are ignored. The efficiency can be increased by eliminating the steps in the traditional approaches, such as isolating, taxonomically differentiating, fermenting, and testing large numbers of randomly selected, mostly non-producing, microorganisms.

The present invention offers simple, but powerful, tools for the formulation and incorporation of diverse media and for inoculation. It also can provides a completely portable system, enabling its use directly in the field.

The present invention also provides an easy and effective means for evaluating the ability of different media, sample types, incubation conditions, etc. to promote and facilitate the detection of microorganisms producing antimicrobial activities of interest. The fact that the method can be portable and used in the field renders it a powerful tool for exploring and evaluating new microbial niches and environments for the presence of new antimicrobial producing microorganisms. It may also find utility in more general studies of microbial ecology.

The present invention has been used to identify fungal, eubacterial and actinomycete antimicrobial producers from natural samples of soil, sediment, plant materials, water, etc. The “hit rate”, the number of wells generally exhibiting antimicrobial activity (vs. S. aureus), using the microcosm approach appears to fall between 3-10% (bacteria ˜3%, fungi ˜6%, actinomycetes ˜10%) and the overall recovery of producing isolates from active microcosms has been observed to be ≧60%. Clearly, the hit rate will be a function of the screening target. Especially for targets with an anticipated low hit rate, this approach should readily provide the high throughput needed to find active cultures.

Furthermore, the present invention can be coupled with sophisticated screens for antimicrobials, in the discovery of microorganisms that produce new and useful antibiotics.

### EXAMPLES

Examples are provided below to further illustrate different features of the present invention. The examples also illustrate useful methodology for practicing the invention. These examples do not limit the claimed invention.

### Materials and Methods

Apparatus

Sterile 96-well filter plates (MultiScreen-BV, MABV S12 10, Millipore) are used to prepare microcosm plates. Brushes (acid brush, 872SO014) are obtained from Techni-Tool. Polyolefin sheets (Gel-Handler, 33-8643-05) are obtained from PGC Scientific. The Tissue Tearor was obtained from Biospec Products and the Autoplate 4000 from Spiral Biotech.

### Example 1

### Preparation of Microcosm Plates

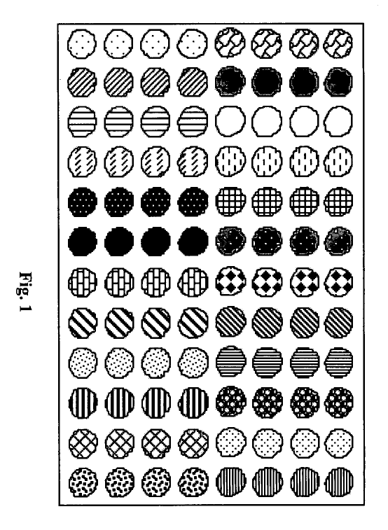

A basal agar medium (275 μl) is dispensed at 75° C. into each well of a filter plate using a Multidrop 384 (ThermoLabSystems). After the basal agar has solidified, liquid (20 μl) solutions of various nutrients may be added simultaneously to each of the wells using a Quadra (TomTec). The addition of selective antibiotics may also be made; however care must be taken that the antibiotics chosen will not interfere with the intended detection system. After addition, the nutrients are allowed to diffuse through the basal agar medium overnight. This also allows time for the liquid to be absorbed. Particulate substrates, e.g. casein, chitin, cellulose, etc. may be sprinkled into appropriate wells. For a 96 well plate, up to 96 different media may be used for each microcosm plate. However, we have found that in practice, an array of 24 different media is more practical (FIG. 1). The choice of nutrients or other additions that are employed may be determined, for example, by the types of microorganisms being sought or the types of natural samples being dealt with and clearly is at the discretion and creativity of the microbiologist.

### Example 2

### Inoculation of Microcosm Plates

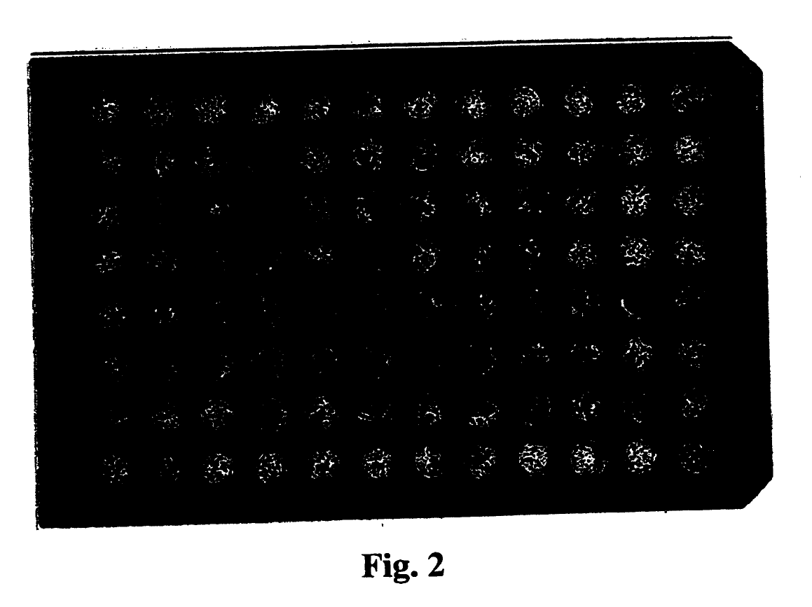

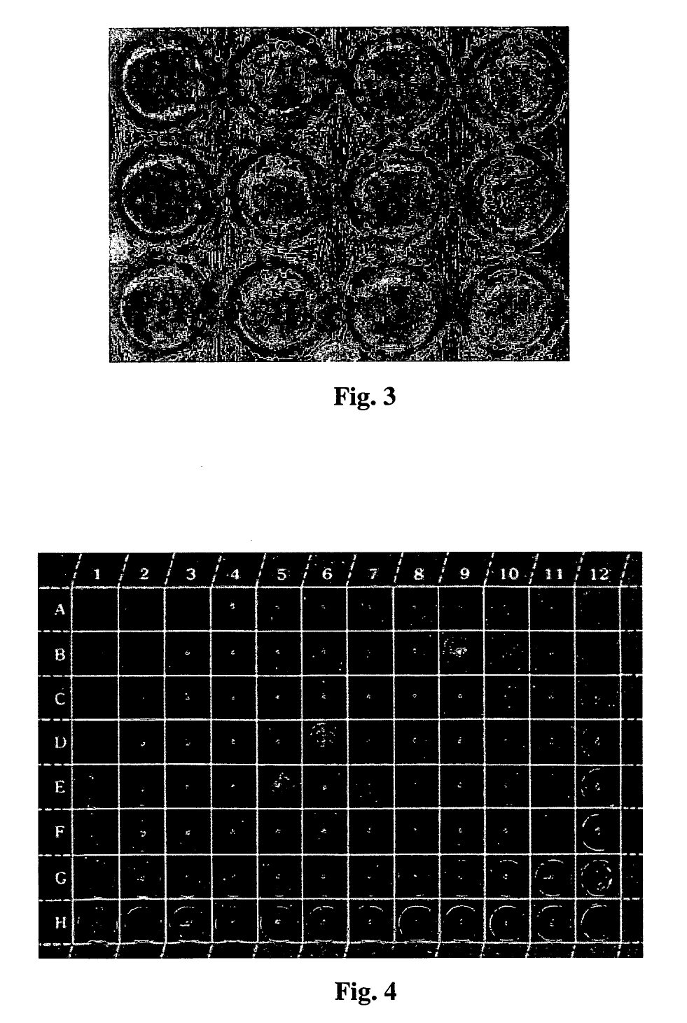

Dry, particulate samples such as soil or finely divided matter can be inoculated by sprinkling the sample directly onto the microcosm plate (FIG. 2). Excess material may be removed from the wells by inverting the plate and sharply tapping it against the palm of the hand. Moist or wet samples, such as soil, sediments, water etc., are easily inoculated using a small, sterile disposable brush. The bristles of the brush are used to pick up the inoculum and then the brush is used to distribute or “paint” (or ‘dab’)the sample over the microcosm plate. The result is that each well is inoculated with particles of the sample (FIG. 3).

Following inoculation, each microcosm plate is placed in a zipper seal bag (01-816-1D, Fischer Sci.) to avoid drying during prolonged incubation. As with media, different types of samples, different sample pre-treatments and different microcosm incubation conditions may be used and are at the discretion of the microbiologist.

### Example 3

### Detection of Active Microcosms

Active microcosms may be distinguished using a suitable agar based microbial detection system. This can be done by first removing the flexible plastic backing from the underside of a microcosm plate to expose the membrane filters closing the bottom of each well. The membrane filter side of the plate then is placed in firm contact with the detection system agar and incubated for a predetermined time interval of 30 minutes to 18 hour at 4° C. to allow diffusion of any antimicrobial activity from the agar in a microcosm well, through the membrane filter and into the underlying detection system agar. The microcosm plate is removed and saved and the microbial detection system is incubated at a temperature permitting growth. Activity is judged based on the appropriate readout for the particular detection system being used, e.g., zones of inhibition. A positive response can then be mapped back to the active well in the microcosm plate from which the producing microorganism(s) may be isolated.

### Example 4

### Isolation and Detection of Antibiotic Producing Cultures from an Active Microcosm

Antibiotic producing culture(s) can be isolated from active microcosms by serial dilution and plating of the microcosm microbial community onto an appropriate agar medium. The agar and associated microorganisms from an active microcosm well are removed using a sterile swab and suspended in 9 ml of an appropriate diluent. A Tissue Tearor is used to macerate the agar and suspend the cells. Ten-fold serial dilutions of this preparation are made and dilutions (10⁻³, 10⁻⁴, and 10⁻⁵) are plated using 150 mm dia. petri plates and an Autoplate 4000.

After incubation, producing colonies present among the isolates are detected by overlaying with a solid agar overlay. Agar overlays are prepared by adding approximately 3 ml of distilled water to the bottom of an empty 150 mm. dia. petri plate. A plastic disk (polyolefin, 150 mm dia.) then is pressed into place against the bottom of the petri plate and held there by a thin layer of distilled water sandwiched between the disk and the plate bottom. Excess water is removed and molten agar (˜30 ml, 50° C.) seeded with an appropriate detection system then is poured into the petri dish and allowed to solidify. The edge of the agar overlay is separated from the sides of the petri plate using a small, pointed spatula. The plastic disk is used to lift the solid agar overlay free from the petri plate. The agar overlay then is applied onto the colonies growing on the isolation plate by sliding it from the supporting plastic disk. A glass or plastic spreader rod is used to press the agar overlay against the colonies and to remove any trapped air to ensure good contact between the overlay and the isolation agar. After incubation, the plate with the overlay is examined for colonies that produce the desired readout, e.g., a zone of inhibition. The colonies are picked from beneath the overlay and purified prior to fermentation.

### Example 5

### Results

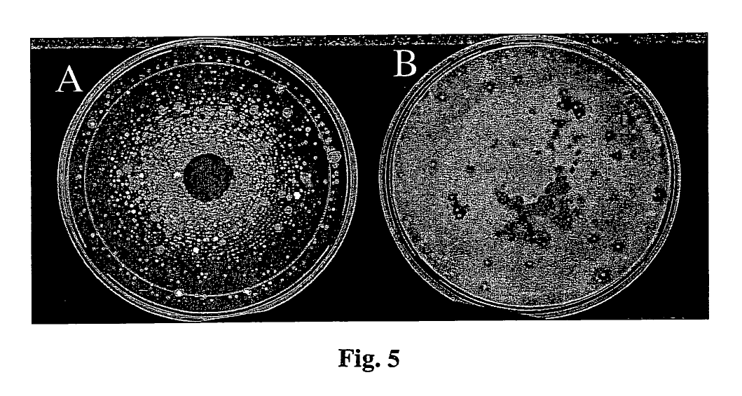

A sample of sand from a salt marsh in southern New Jersey was applied to a microcosm plate (FIG. 2) containing 24 different media arrayed as shown in FIG. 1. The plate was incubated for 10 days at 25° C. and then assayed for antimicrobial activity against Staphylococcus aureus. As shown in FIG. 4, approximately one-third (27) of the microcosms were observed to be “active” in that they produced distinct zones of inhibition. The microorganisms from active microcosms were plated on marine agar media (FIG. 5A). Antimicrobial producing colonies were detected by overlaying with S. aureus seeded agar. Antibiotic producing colonies were observed to produce zones of inhibition (FIG. 5B). These colonies then were isolated, purified and subsequently tested in other target based antimicrobial screens.

Other embodiments are within the following claims. While several embodiments have been shown and described, various modifications may be made without departing from the spirit and scope of the present invention.

1. A method of screening microorganisms, comprising,
inoculating to media a sample containing a plurality of different microorganisms,
incubating the inoculated media, and
detecting the presence of microorganisms having desired characteristics on the incubated medium with a reporter system.

2. The method of claim 1 further comprising isolating the detected microorganisms.

3. The method of claim 1 wherein the desired characteristics include producing antimicrobial.

4. The method of claim 1 wherein the sample is selected from the group consisting of soil, litter, and water.

5. The method of claim 1 wherein the media are agar nutrient media.

6. The method of claim 5 wherein agar nutrient media are different agar nutrient media.

7. The method of claim 1 wherein the media comprise a basal salts mineral medium supplemented with a carbon and/or nitrogen source.

8. The method of claim 1 wherein the media are contained in the wells of a 96-well membrane filter plate.

9. The method of claim 3 wherein the media are contained in a plate with a membrane filter side.

10. The method of claim 3 wherein the presence of microorganisms producing antimicrobial is detected by placing the membrane filter side with a reporter system.

11. The method of claim 10 wherein the reporter system is agar seeded with a microorganism targeted by the antimicrobial.

12. The method of claim 10 wherein the reporter system is agar seeded with Staphylococcus aureus.
